(19) United States
(12) Patent Application Publication
(10) Pub. No.: US 2006/0051409 A1
(43) Pub. Date: Mar. 9, 2006

(54) Bezonatate liquid hard capsule dosage form

(76) Inventor: Pieter J. Groenewoud, Powder Springs, GA (US)

Correspondence Address: ARNALL GOLDEN GREGORY, LLP, 171 17TH STREET, SUITE 2100, ATLANTA, GA, 30363, US

(21) Appl. No.: 10/936,378

(22) Filed: Sep. 8, 2004

(51) Int. Cl. A61K 9/64

(52) U.S. Cl. 424/456

(57) ABSTRACT

A pharmaceutical dosage includes a hard capsule, benzonatate and a sealer ring. The hard capsule has a bottom half and a top half that covers the bottom half. The benzonatate is in liquid form and is placed in the bottom half of the hard capsule. The sealer ring seals the top half to the bottom half.

### BACKGROUND OF THE INVENTION

1. Field of the Invention

The present invention relates to pharmaceutical dosages and, more specifically, to a pharmaceutical dosage that includes benzonatate in a hard capsule.

2. Description of the Prior Art

Benzonatate, a known composition used as an antitussive, is manufactured in liquid form. Typically, dosages of benzonatate are placed in a soft gel cap. Some patients, especially children, have a tendency to bite into soft gel caps, expecting them to taste like candy. When bitten hard enough, the liquid in the gel cap is released into the patient's mouth. Benzonatate causes local anesthesia in the mouth and throat. This local anesthesia can lead to choking in certain patients. Therefore, there is a need for benzonatate in a form with a reduced likelihood of being released in the patient's mouth.

Also, gel caps are relatively expensive to produce, requiring solvents to provide the proper finish. Typical solvents used include methylene chloride, which is an undesirable material in the finished gel cap and may have undesirable environmental consequences.

Therefore, there is a need for a benzonatate dosage form that has a lower likelihood of being bitten into.

### SUMMARY OF THE INVENTION

The disadvantages of the prior art are overcome by the present invention which, in one aspect, is a pharmaceutical dosage that includes benzonatate, in liquid form, disposed in a hard capsule.

In another aspect, the invention is a pharmaceutical dosage that includes a hard capsule, benzonatate and a sealer ring. The hard capsule has a bottom half and a top half that covers the bottom half. The benzonatate is in liquid form and is disposed in the bottom half of the hard capsule. The sealer ring seals the top half to the bottom half.

In yet another aspect, the invention is a method of making a pharmaceutical dosage, in which liquid benzonatate is placed in a hard capsule. The hard capsule is sealed with the liquid benzonatate therein.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a side elevational view of one illustrative embodiment of the invention.

FIG. 2 is a side elevational view of a second illustrative embodiment of the invention.

### DETAILED DESCRIPTION OF THE INVENTION

A preferred embodiment of the invention is now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

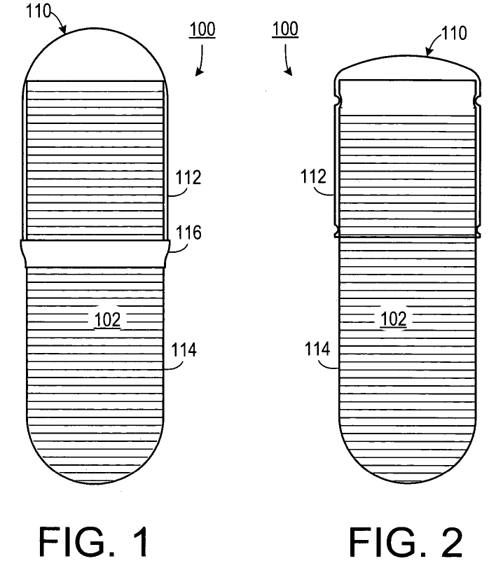

As shown in FIGS. 1 and 2, a pharmaceutical dosage 100 that discourages biting includes a hard capsule 110 and liquid form benzonatate 102 that is placed in the hard capsule 110. The hard capsule 110 includes a bottom half 114, into which the benzonatate is disposed and a top half 112, that covers a portion of the bottom half 114.

A sealer ring 116 may be used to seal the top half 112 to the bottom half 114. One type of sealing machine that may be used to apply the sealer ring includes the S-40 HICAPSEAL, available from Shionogi Qualicaps, Inc., 6505 Franz Warner Parkway, Whitsett, N.C. 27377. As is generally known in the pharmaceutical arts, the hard capsule 110 could be made from materials such as: gelatin, gelatin and polyethylene glycol, and hypromellose.

The above described embodiments are given as illustrative examples only. It will be readily appreciated that many deviations may be made from the specific embodiments disclosed in this specification without departing from the invention. Accordingly, the scope of the invention is to be determined by the claims below rather than being limited to the specifically described embodiments above.

What is claimed is:

1. A pharmaceutical dosage, comprising:
a. a hard capsule; and
b. benzonatate, in liquid form, disposed in the hard capsule.

2. The pharmaceutical dosage of claim 1, wherein the hard capsule comprises:
a. a bottom half, into which the benzonatate is disposed;
b. a top half, that covers a portion of the bottom half;
c. a sealer ring that seals the top half to the bottom half.

3. The pharmaceutical dosage of claim 1, wherein the hard capsule comprises gelatin.

4. The pharmaceutical dosage of claim 1, wherein the hard capsule comprises gelatin and polyethylene glycol.

5. The pharmaceutical dosage of claim 1, wherein the hard capsule comprises hypromellose.

6. A pharmaceutical dosage, comprising:
a. a hard capsule, having a bottom half and a top half that covers a portion of the bottom half;
b. benzonatate, in liquid form, disposed in the bottom half of the hard capsule; and
c. a sealer ring that seals the top half to the bottom half.

7. A method of making a pharmaceutical dosage, comprising the steps of:
a. placing liquid benzonatate in a hard capsule; and
b. sealing the hard capsule with the liquid benzonatate therein.

8. The method of claim 7, wherein the sealing step comprises applying a sealer ring to the capsule.
